(19) United States
(12) Patent Application Publication
(10) Pub. No.: US 2021/0299422 A1
(43) Pub. Date: Sep. 30, 2021

(54) Needle Penetration Preparation Device And Related Systems And Methods

(71) Applicant: COMBOSTRIKE, Santa Cruz, CA (US)

(72) Inventors: Lucas McLelan, Santa Cruz, CA (US); Benjamin Moga, Soquel, CA (US)

(21) Appl. No.: 17/264,364

(22) PCT Filed: Jul. 30, 2019
(86) PCT No.: PCT/US19/44022

§ 371 Date: Jan. 29, 2021

Related U.S. Application Data
(60) Provisional application No. 62/711,839, filed on Jul. 30, 2018.

(51) Int. Cl. A61F 13/40; A61M 5/42

(52) U.S. Cl. CPC A61M 35/006; A61M 5/427; A61M 2205/75; A61M 2205/584; A61M 2205/581

(57) ABSTRACT

Disclosed herein are various needle penetration preparation devices and methods in which each of the devices have a skin marking mechanism at one end of the device body, a sterilization applicator at another end of the device body, and a compromisable sterilization solution reservoir adjacent to the sterilization applicator.

### CROSS-REFERENCE TO RELATED APPLICATION(S)

This application claims priority to International PCT Application No. PCT/US19/44022, filed on Jul. 30, 2019, which claims benefit under 35 U.S.C. § 119(e) to U.S. Provisional Application 62/711,839, filed Jul. 30, 2018 and entitled “Needle Penetration Preparation Device and Related Systems and Methods,” which is hereby incorporated herein by reference in its entirety.

### FIELD

The various embodiments herein relate to needle-delivered medical treatment methods and devices, and more specifically to patient needle penetration site preparation methods and devices.

### BACKGROUND OF THE INVENTION

Venipuncture used for purposes of intravenous (“IV”) cannulation and drawing blood is often performed incorrectly due to the technician (1) insufficiently sterilizing and prepping the puncture site, (2) touching and thereby compromising the site to find the target after it has been sterilized, or (3) a combination of the two. These errors can lead to a possible blood infection in the patient and/or inaccurate lab results from a cross-contaminated specimen.

There is a need in the art for improved methods and devices for identifying and preparing a needle penetration site on a patient's skin.

### BRIEF SUMMARY

Discussed herein are various devices and methods for the preparation of a puncture site on a skin area of a patient.

In Example 1, a puncture preparation device comprises a body comprising at least one flexible portion, a skin marking device disposed at a first end of the body, a sterilization applicator disposed at a second end of the body, and a compromisable sterilization solution reservoir disposed in the body, wherein the reservoir is adjacent to the sterilization application.

Example 2 relates to the puncture preparation device according to Example 1, wherein the at least one flexible portion is disposed between a first portion of the body and a second portion of the body, wherein the first portion can be moved from a first position into a second position in relation to the second portion of the body.

Example 3 relates to the puncture preparation device according to Example 2, wherein the movement of the first portion into the second position causes the compromisable sterilization solution reservoir to break such that sterilization solution disposed therein can escape.

Example 4 relates to the puncture preparation device according to Example 1, further comprising a removable cap disposable over the skin marking device.

Example 5 relates to the puncture preparation device according to Example 4, wherein the removable cap comprises a skin marking feature disposed thereon.

Example 6 relates to the puncture preparation device according to Example 1, further comprising a filter disposed between the compromisable sterilization solution reservoir and the sterilization applicator.

Example 7 relates to the puncture preparation device according to Example 1, further comprising a solution presence indicator operably coupled to the sterilization applicator.

In Example 8, a puncture preparation device comprises a body, a skin marking mechanism disposed at a first end of the body, a removable cover removably disposable over the skin marking device, the removable cover comprising a skin marking feature disposed thereon, a sterilization application structure disposed at a second end of the body, and a fracturable sterilization solution reservoir disposed in the body.

Example 9 relates to the puncture preparation device according to Example 8, wherein the body comprises at least one flexible portion disposed between a first portion of the body and a second portion of the body, wherein the first portion is movable relation to the second portion of the body at the at least one flexible portion.

Example 10 relates to the puncture preparation device according to Example 9, wherein movement of the first portion in relation to the second portion causes the fracturable sterilization solution reservoir to break such that sterilization solution disposed therein can flow into the sterilization application structure.

Example 11 relates to the puncture preparation device according to Example 9, further comprising an actuation mechanism operably coupled with the body, wherein actuation of the actuation mechanism causes the fracturable sterilization solution reservoir to fracture.

Example 12 relates to the puncture preparation device according to Example 8, wherein the skin marking feature comprises at least one raised feature on a distal end of the removable cover.

Example 13 relates to the puncture preparation device according to Example 12, wherein the at least one raised feature is configured to create an indentation in a patient's skin when sufficient force is applied.

Example 14 relates to the puncture preparation device according to Example 8, further comprising a solution presence indicator operably coupled to the sterilization application structure.

Example 15 relates to the puncture preparation device according to Example 8, further comprising a filter disposed between the fracturable sterilization solution reservoir and the sterilization application structure.

In Example 16, a method of preparing a target site on a patient's skin for a needle penetration comprises positioning a needle penetration preparation device with a single hand of a user such that a skin marking mechanism is disposed in proximity with the target site. The device comprises a device body, the skin marking mechanism disposed at a first end of the device body, a removable cap removably coupled to the skin marking device, the removable cap comprising a skin marking feature disposed on the removable cap, a sterilization applicator disposed at a second end of the device body, and a sterilization solution reservoir disposed in the body, wherein the reservoir is adjacent to the sterilization applicator. The method further comprises marking the target site with the skin marking mechanism or the skin marking feature, repositioning the device within the single hand of the user such that the sterilization applicator is disposed in proximity with the target site, causing the sterilization solution reservoir to be compromised such that the sterilization solution flows out of the reservoir and into the sterilization applicator, and applying the sterilization solution to the target site with the sterilization applicator.

Example 17 relates to the method according to Example 16, wherein the causing the sterilization solution reservoir to be compromised further comprises deforming the device body such that the reservoir is compromised.

Example 18 relates to the method according to Example 16, wherein the causing the sterilization solution reservoir to be compromised further comprises actuating an actuation mechanism such that the reservoir is compromised.

Example 19 relates to the method according to Example 16, further comprising removing the removable cap from the skin marking mechanism prior to marking the target site with the skin marking mechanism.

Example 20 relates to the method according to Example 16, wherein the applying the sterilization solution further comprises applying the sterilization solution for a minimum amount of time based on an indicator operably coupled to the sterilization applicator.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a perspective view of a target skin site preparation device, according to one embodiment.

FIG. 2 is a perspective view of another target skin site preparation device, according to another embodiment.

FIG. 3 is a perspective view of a protective cover, according to one embodiment.

FIG. 4 is a side view of a target skin site preparation device being deformed by a user, according to one embodiment.

FIG. 5A is a side view of a further target skin site preparation device, according to another embodiment.

FIG. 5B is a cross-sectional view of the target skin site preparation device of FIG. 5A, according to one embodiment.

FIG. 6 is a side view of another target skin site preparation device, according to another embodiment.

FIG. 7 is a side view of a further target skin site preparation device, according to a further embodiment.

### DETAILED DESCRIPTION

The various embodiments herein relate to a device that can 1) mark the target area of the skin of the patient prior to venipuncture, and 2) sterilize the target skin surface in preparation for needle penetration. The device implementations as disclosed or contemplated herein can reduce the incidence of improper blood collection techniques by allowing the target site to be marked before being prepped and eliminating re-touching of the site after it is cleaned. Further, certain embodiments can also include the use of a timer to ensure that the target site is prepped for the correct length of time. In addition, as will be described in additional detail herein, various implementations allow for use of the device implementations with only one hand. Drawing blood is an active and involved process such that the ability to keep one hand free during the marking and sterilizing process is of great value.

In certain implementations, the various device embodiments herein can be used by a healthcare professional to mark the skin, thereby identifying or targeting a location for blood collection, and also to sterilize the skin. The device can be used by the professional to mark and sterilize the skin for herself/himself or for another professional. For example, a phlebotomist may use a device as disclosed or contemplated herein to locate and mark a vein in the patient's arm for the phlebotomist herself or for another healthcare professional.

The intended users of the various embodiments herein can include any healthcare professionals, including, but not limited to, doctors, anesthesiologists, nurses, paramedics, respiratory therapists, and phlebotomists. Further, the various methods and devices disclosed or contemplated herein can be used for, but are not limited to, IV cannulation, arterial blood gas draw, lumbar punctures, peripherally inserted central venous catheter (“PICC”) insertion, ultrasound guided IV starts, central line placement, arterial line placement, blood cultures and basic blood draws.

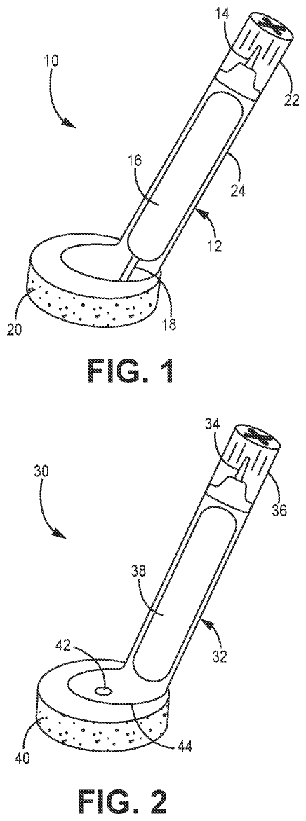

One embodiment of a target skin site preparation device 10 is depicted in FIG. 1. The device 10 has a body 12, a skin marking device 14, a sterilizing solution reservoir 16 that is configured to hold sterilizing solution, a channel 18 fluidically coupled to the reservoir 16, and a sterilization pad 20 coupled to a distal end of the body 12 and adjacent to the channel 18. In addition, this implementation has a removable cap 22 that can be removably placed over the marking device 14 to cover the marking device 14 while it is not in use. Further, in this device 10 embodiment, the skin marking device 14 is disposed at one end of the body 12 and the sterilization pad 20 is disposed at the other, such that the user can operate the device 10 using one end and then the other as described in further detail below. It is understood that any other embodiment disclosed or contemplated herein can also have the marking device and sterilization pad at opposing ends of the body.

In this embodiment, the device 10 has no protruding components or mechanisms. That is, the body 12 has a surface as shown that is uninterrupted along its length by any protruding features of any kind. As such, the protrusion-less body 12 facilitates the device 10 being operated with one hand by a user, as will be described in additional detail below.

According to this exemplary embodiment, the skin marking device 14 can be a marking pen 14 or any other device or apparatus for marking a target site of a patient's skin. In one implementation, the marking pen 14 utilizes marker ink to mark the skin that is neither permanent nor toxic. In one embodiment, the ink is a known marker ink for purposes of marking a patient's skin for medical purposes. Further, the ink can be chosen for any one or more of the following characteristics: (1) ability to remain on the patient's skin and visible during and after skin sterilization; (2) an optimal color for being visible on a patient's skin; and/or (3) length of time that the ink remains visible to the human eye. Alternatively, the marking pen 14 can use a known dye having any of the same or similar characteristics.

It is understood that the skin marking device incorporated into any embodiment disclosed or contemplated herein can be any of the specific exemplary skin marking mechanisms described above having any of the features thereof.

In this particular implementation as shown in FIG. 1, the reservoir 16 is a breakable or otherwise compromisable container 16 that can be broken by applying force at an appropriate point along the length of the body 12. The application of force causes the body 12 to bend, thereby breaking the container 16. For example, in one embodiment as best shown in FIG. 4 and discussed in further detail below, a user grips the body 12 (or, in FIG. 4, the body 72, or any other body of any other device disclosed or contemplated herein) in the user's hand 74 with the user's four fingers 76 as shown such that the user's thumb 78 can be used to apply radially-directed force to the side of the body 12 in the direction of arrow A at a predetermined point along the length of the body 12. In this implementation, the body 12 has a flexible portion along a length of the body 12 such that application of force by a user's thumb 78 (or any other known way to apply such force at such a location) causes the body 12 to bend. The bending of the body 12 causes a wall 24 of the body 12 to come into contact with the container 16 such that sufficient force applied by the thumb 78 causes the wall 24 to break the container 16.

The breaking of the container 16 causes the container 16 to be compromised such that sterilization fluid previously contained within the container 16 is released onto the sterilization pad 20 via the channel 18. Alternatively, it is understood that the term “compromisable,” “breakable,” and “fracturable” are intended in this application to encompass any other known release mechanism that can be used to break the container 16 or to otherwise release or provide sterilization fluid to the sterilization pad 20 from the container 16. It is understood that the specific reservoir incorporated into any embodiment disclosed or contemplated herein can have any of the specific exemplary reservoir features or structures described in this paragraph.

In one embodiment, the sterilization pad 20 (or any other sterilization pad in any other device implementation disclosed or contemplated herein) is any known structure 20 for use in retaining at least some sterilization solution and applying the solution to a target skin site prior to needle penetration. For example, in one specific implementation, the pad 20 can be made of a porous material, such as a sponge or other sponge-like material. Alternatively, the pad 20 can have any known structure and/or be made of any known material for use in a sterilization pad or application surface.

According to certain specific implementations in which the reservoir is a fracturable reservoir, the sterilization pad (or any other sterilization pad in any other device implementation disclosed or contemplated herein) can be made of a porous material that acts as a filter to catch any small pieces of fractured reservoir materials that may splinter off during the fracturing event. This can prevent or reduce the chances of foreign materials from the reservoir falling on the skin as part of the sterilization process.

In use, the configuration of the device 10 (and all other embodiments disclosed or contemplated herein) allows a healthcare professional to both mark the skin and sterilize the skin using the same device 10. One advantage of this device 10 (and the other device embodiments herein) is that the dual-functionality of this device 10 to perform both the marking and sterilization function can be performed by a user utilizing only one hand, rather than two. That is, the configuration of the device 10 allows a user to easily reposition the device 10 in the user's single hand from the marking position (in which the skin marking device 14 is positioned to make contact with the target area of the patient's skin) to the sterilization position (in which the sterilization pad 20 is positioned to make contact with the target area). Further, the protrusion-less body 12 of the exemplary device 10 can further facilitate the repositioning of the device 10 in a user's hand by eliminating the possibility that a protruding portion of the body 12 may make inadvertent contact with the hand during repositioning and thus adversely impact that repositioning, including potentially by causing the device 10 to be dropped as a result.

Turning to the specific steps of marking and sterilization, the user first removes the cap 22 from the skin marking device 14 and holds the device 10 to utilize the skin marking device 14 to mark the skin of the patient at the desired target needle penetration location. Once the target location has been identified (or “marked”) by the marking device 14, the user can then utilize the device 10 to sterilize the target location. More specifically, the user can first cause sterilization fluid to flow into or otherwise be positioned in the sterilization pad 20, and then place the sterilization pad 20 into contact with the target puncture location to sterilize the location. As discussed above, in the device 10 embodiment disc, the user causes the sterilization fluid to flow into the sterilization pad 20 by breaking the container 16 in the fashion described above and depicted in FIG. 4. Alternatively, any other device using any other method of releasing the sterilization fluid can be used. Once the sterilization fluid is released into the pad 20, the device 10 is repositioned in the user's hand to utilize the sterilization pad 20. That is, the user positions the device 10 in the user's hand such that the sterilization pad 20 is positioned to be placed in contact with the skin of the target area, thereby allowing for the user to sterilize the area with the pad 20.

Another embodiment of a target skin site preparation device 30 is depicted in FIG. 2. The device 30 has a body 32, a skin marking device 34, a removable cap 36 removably disposed over the marking device 34, a sterilization solution reservoir 38 that is configured to hold sterilization solution, a sterilization pad 40 disposed at an end of the body 32 opposite the skin marking device 34, an indicator 42 disposed on the body 32, and a filter 44 disposed between the reservoir 38 and the pad 40.

The components of FIG. 2 with the same or similar names as the equivalent components of FIG. 1 are substantially the same as those equivalent components except as noted herein. The indicator 42 in this implementation can be any known form of indicator for indicating that sterilization solution has been released into or is otherwise present in the pad 40. According to one specific embodiment, the indicator 42 can be a visual indicator 42 that is made of any known material that can change color when contacted by a known sterilization solution. In this implementation, the indicator 42 is in fluidic communication with the pad 40 such that any sterilization solution that is present in the pad 40 also makes contact with the indicator 42, thereby causing the indicator 42 to change color and thus provide a notification to the user that the pad 40 contains the sterilization solution. In certain embodiments, the indicator 42 is in fluidic communication with the pad 40 via a wicking component (also referred to herein as a “indicator wick”) (not shown). As such, when sterilization solution is added or released to the pad 40, the solution contacts the indicator wick (not shown) after a predetermined period of time, which causes the solution to be transported along the wicking component (not shown) to the indicator 42 in a predetermined period of time, thereby causing the indicator 42 to change color, which visually alerts the user that the predetermined amount of time has passed during which the sterilization solution has been present in the pad 40. In one embodiment, the predetermined period of time between the solution being released into the pad 40 and the indicator 42 changing color is about 30 seconds. Alternatively, it can be any predetermined amount of time. As such, the user can utilize the indicator 42 to apply the sterilization solution to the target area of the patient's skin for a predetermined amount of time.

The reason for the alert after a predetermined period of time is that it is understood that successful skin sterilization depends on the amount of time that the target skin area is scrubbed or otherwise treated with the sterilization solution prior to needle penetration. Generally, the recommended time is at least around thirty seconds, although the alert embodiments disclosed or contemplated herein are not limited to any specific time period. Thus, various alternative implementations of the devices disclosed or contemplated herein can provide an alert that encourages the user to scrub or otherwise treat the target skin site for a recommended amount of time that is indicated by the alert.

In various alternative implementations, the indicator can provide an audible alert, a visual indicator, or any other known form of alert.

The filter 44 mentioned above is disposed between the reservoir 38 and the sterilization pad 40 such that when the sterilization fluid is released from the reservoir 38 via any method or mechanism disclosed or contemplated herein, the fluid must flow through the filter 44 as it travels from the reservoir 38 to the pad 40. As such, the filter 44 is any known filter 44 that allows the passage of a known sterilization solution while trapping any particles therein, including, for example, any pieces of glass (or any other known material of which the reservoir may be made) from the broken or compromised reservoir. Thus, the filter 44 helps to prevent the passage of any broken pieces of glass or other such material onto the skin of the patient by trapping the material in the filter 44.

It is understood that any indicator 42 embodiment and any filter 44 embodiment as described above can be incorporated into any device embodiment disclosed or contemplated herein.

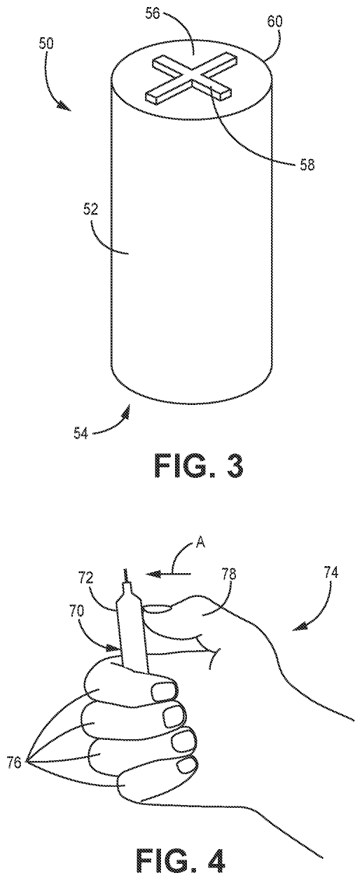

One exemplary embodiment of a removable cap 50 that can be used with any device implementation disclosed or contemplated herein is depicted in FIG. 3. In this implementation, the cap 50 has a cap body 52 having an opening 54 at its proximal end that is configured to receive the marking device of any embodiment herein and be positioned thereover. Further, the body 52 has a distal end 56 with a protruding (or “raised”) feature 58 disposed thereon, along with a ridge 60 disposed around the outer circumferential edge of the distal end 56 that protrudes from the distal end 56 to a height that is substantially the same as the raised feature 58. In one embodiment, the raised feature 58 is shaped like a cross or “X” as shown. Further, according to one implementation, the ridge 60 has a width ranging from about 2 mm to about 15 mm. Alternatively, the ridge 60 can have any known width within the parameters of the cap 50.

In use, the raised feature 58 and the ridge 60 can be used to mark the target skin on a patient via sustained pressure by the user. That is, the cap 50 remains disposed on the device and the device is positioned such that the distal end 56 of the cap 50 is pressed against the target area of the patient's skin with sustained pressure for a predetermined period of time such that raised feature 58 and the ridge 60 create an indentation shaped like the raised feature 58 and the ridge 60 in the patient's skin for some period of time after the device is retracted. As such, the cap 50 can be used to mark the target area of the skin instead of any marking device embodiment as described elsewhere herein. In the specific exemplary implementation as shown, the raised feature 58 and the ridge 60 are shaped such that the resulting mark on the skin is a ring with a cross in the middle, thereby creating a good “bulls-eye”-like target on the patient's skin for puncture with a needle. Alternatively, any raised feature 58 and/or ridge 60 of any shape or other physically protruding features at the distal end 56 of the cap 50 can be used. It is understood that the various steps for using the cap 50 for marking purposes are substantially similar to the steps described above with respect to the marking device embodiments, except that the cap 50 is not removed from the device and instead remains disposed over the marking device.

In one embodiment, the raised feature 58 and the ridge 60 on the cap 50 are used to mark the patient instead of the marking device in those circumstances where the marking device does not work very well. For example, in those situations where ultrasound gel has been placed on the patient's skin, the ink from the marking device can smear and thus fail to mark the target site sufficiently to allow a user to see the mark and thereby successfully puncture the skin at the target site. In contrast, the use of the raised feature 58 and the ridge 60 creates a physical mark in the form of an indentation that does not require ink and thus cannot smear or otherwise fail to mark the target site. Thus, the cap 50 with the raised feature 58 and ridge 60, according to certain implementations, can be useful for ultrasound-guided venipuncture procedures such as ultrasound-guided IVs, PICC line placement, or central line placement. Alternatively, the cap 50 can be useful in any known procedure in which marking a target site can be helpful.

FIG. 4, as discussed above in the context of device 10, displays one method of breaking a sterilization solution container within a device 70 such that the solution is deployed into the sterilization pad (not shown), according to one embodiment. That is, because the device 70 (and all other device embodiments disclosed or contemplated herein) is intended for one-handed use, the body 72 must have a flexible portion as discussed above to allow for bending the body 72 while holding the device 70 in one hand such that the solution reservoir (not shown) can be broken, thereby releasing the solution. In this exemplary implementation, the device 70 is gripped by the four fingers 76 of the hand 74 such that the position of the device 70 can be maintained while the thumb 78 of the user applies pressure in the direction of arrow A to the body 72, thereby causing the body 72 to bend and thus causing the reservoir (not shown) within the body 72 to break. Alternatively, any known method of breaking the reservoir (not shown) by bending the body 72 can be used. It is understood that this method can be used with any device embodiment disclosed or contemplated herein having a flexible body.

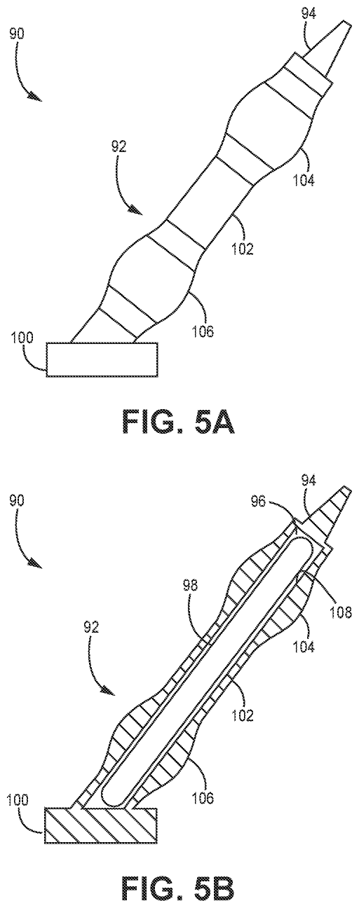

Another embodiment of a target skin site preparation device 90 is depicted in FIGS. 5A and 5B. The device 90 has a body 92, a skin marking device 94, an inner cavity 96 defined within the body 92 (as best shown in FIG. 5B), a sterilization solution reservoir 98 disposed within the cavity 96 (as best shown in FIG. 5B) that is configured to hold sterilization solution, and a sterilization pad 100 disposed at an end of the body 92 opposite the skin marking device 94. In addition, the device 90 can also have a removable cap (not shown) according to any cap implementation disclosed or contemplated herein.

The components of FIGS. 5A and 5B with the same or similar names as the equivalent components of FIGS. 1 and 2 are substantially the same as those equivalent components except as noted herein.

In this exemplary implementation as shown in FIGS. 5A and 5B, the body 92 is ergonomically designed as shown for easy holding and handling by the health professional and easy manipulating of the device 90 to mark the patient's skin and then sterilize that area in the manner described elsewhere herein. Further, the body 92 has a grip 102 that is designed and strategically positioned on the body 92 to enhance the gripability of the body 92 by the user. More specifically, the grip 102 is a length of the body 92 that has a smaller circumference than the “bulbs” 104, 106 on opposing ends of the grip 102, thereby facilitating a user's ability to grasp and hold onto the grip 102. In certain implementations, the grip 102 enables firm grasping of the body 92 during use, including enabling a smooth, one-handed transition when repositioning the device 90 for use of the marking device 94 or for use of the sterilization pad 100. In other words, the shape of the body 92 and the grip 102 allows the user to easily “flip” or otherwise reposition the device 90 in the user's single hand without having to use a second hand. It is understood that the specific body 92 (and any grip such as grip 102 included thereon) incorporated into any embodiment disclosed or contemplated herein can be any of the specific exemplary body and/or grip configurations described in this paragraph having any of the features thereof.

In addition, the body 92 is also shaped and configured for easy one-handed bending of the body 92 to break the sterilization solution reservoir 98 therein in a fashion similar to various embodiments discussed above. In this particular implementation, at least a portion of the body 92 along its length is flexible. More specifically, in one implementation, the body 92 is flexible at or near one or both of the bulbs 104, 106. As such, in use, imagining the device 90 being incorporated into FIG. 4 as discussed above, a user can grip the body 92 in the user's hand 74 with the user's four fingers 76 at the grip 102 such that the user's thumb 78 can be used to apply radially-directed force to one of the bulbs 104, 106 in the direction of arrow A. The application of force to either bulb 104, 106 by the thumb 78 (or any other known way to apply such force at such location) causes the body 92 to bend, thereby ultimately breaking the reservoir 98 therein. More specifically, as the body 92 bends or otherwise deforms under the force applied by the user, the inner wall 108 of the cavity 96 comes into contact with and applies force to the reservoir 98. As the deformation (bending) continues, the force applied by the inner wall 108 causes the reservoir 98 to be compromised. That is, the wall 108 is urged into and through the reservoir 98 with such force that the reservoir 98 breaks or otherwise is compromised such that the solution therein can escape and flow into the pad 100 via the cavity 96.

The device 90 can also have an indicator or a filter according to any embodiment thereof as described elsewhere herein.

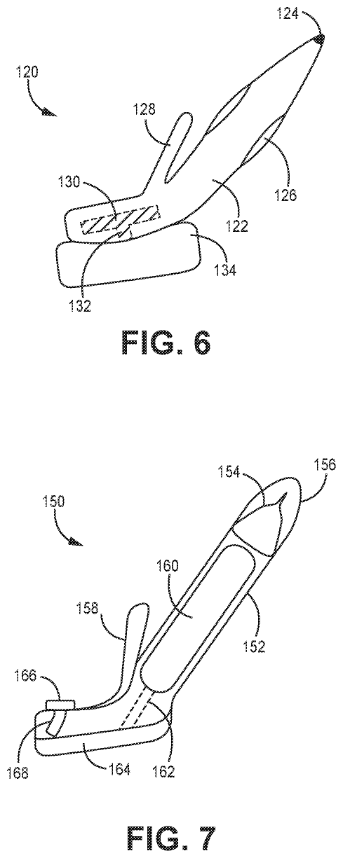

A further implementation of a target skin site preparation device 120 is depicted in FIG. 6. The device 120 has a body 122, a skin marking device 124, a gripping component (also referred to herein as a “grip”) 126, an actuation component 128, a sterilizing solution reservoir 130 that is configured to hold sterilizing solution, a channel 132 fluidically coupled to the reservoir 130, and a sterilization pad 134 coupled to a distal end of the body 122 and adjacent to the channel 132.

The components of FIG. 6 with the same or similar names as the equivalent components of FIGS. 1, 2, and/or 5 are substantially the same as those equivalent components except as noted herein. The actuation component 128 is operably coupled to the reservoir 130 and, in this specific embodiment, is an actuation lever 128. Alternatively, the actuation component 128 can be a button, a knob, any other twist actuator, a pressure actuator, or any other known actuation mechanism for actuating the release or provision of the sterilization fluid as disclosed or contemplated herein. It is understood that the specific actuation component incorporated into any embodiment disclosed or contemplated herein can be any of the specific exemplary actuation mechanisms described in this paragraph having any of the features thereof. Similarly, the specific grip incorporated into any embodiment disclosed or contemplated herein can be any of the specific exemplary gripping components described in this paragraph having any of the features thereof.

In this particular implementation as shown, the reservoir 130 is a breakable or otherwise compromisable container 130 that can be broken by actuation of the actuation component 128 such that sterilization fluid is released onto the sterilization pad 134 via the channel 132. Alternatively, any other known release mechanism can be used in conjunction with the actuation component 128 to release or otherwise provide sterilization fluid to the sterilization pad 134. It is understood that the specific reservoir incorporated into any embodiment disclosed or contemplated herein can have any of the specific exemplary reservoir features or structures described in this paragraph.

Yet another embodiment of a target skin site preparation device 150 is depicted in FIG. 7. The device 150 has a body 152, a skin marking device 154, a removable cap 156 removably disposed over the marking device 154, an actuation component 158, a sterilization solution reservoir 160 that is configured to hold sterilization solution, a channel 162 fluidically coupled to the reservoir 160, a sterilization pad 164 coupled to a distal end of the body 152 and adjacent to the channel 162, and a visual indicator 166 fluidically coupled to a wicking component 168, which is fluidically coupled to the pad 164.

The components of FIG. 2 with the same or similar names as the equivalent components of FIGS. 1, 2, 5, and/or 6 are substantially the same as those equivalent components except as noted herein. The visual indicator 166 is similar to the indicator embodiments disclosed or contemplated elsewhere herein and can be any known material that can change color when contacted by a known sterilization solution. The wicking component 168 and the visual indicator 166 are configured to receive sterilization solution and cause the indicator 166 to change color at a predetermined amount of time after the solution is provided to the pad 164. That is, when sterilization solution is added or released to the pad 164, the solution contacts the indicator wick 168 after a predetermined period of time, which causes the solution to be transported along the wicking component 168 to the indicator 166 in a predetermined period of time, thereby causing the indicator 166 to change color, which visually alerts the user that the predetermined amount of time has passed. In one embodiment, the predetermined period of time between the solution being released into the pad 164 and the indicator 166 changing color is about 30 seconds. Alternatively, it can be any predetermined amount of time.

Although the various embodiments have been described with reference to preferred implementations, persons skilled in the art will recognize that changes may be made in form and detail without departing from the spirit and scope thereof.

What is claimed is:

1. A puncture preparation device comprising:
(a) a body comprising at least one flexible portion;
(b) a skin marking device disposed at a first end of the body;
(c) a sterilization applicator disposed at a second end of the body; and
(d) a compromisable sterilization solution reservoir disposed in the body, wherein the reservoir is adjacent to the sterilization applicator.

2. The puncture preparation device of claim 1, wherein the at least one flexible portion is disposed between a first portion of the body and a second portion of the body, wherein the first portion can be moved from a first position into a second position in relation to the second portion of the body.

3. The puncture preparation device of claim 2, wherein the movement of the first portion into the second position causes the compromisable sterilization solution reservoir to break such that sterilization solution disposed therein can escape.

4. The puncture preparation device of claim 1, further comprising a removable cap disposable over the skin marking device.

5. The puncture preparation device of claim 4, wherein the removable cap comprises a skin marking feature disposed thereon.

6. The puncture preparation device of claim 1, further comprising a filter disposed between the compromisable sterilization solution reservoir and the sterilization applicator.

7. The puncture preparation device of claim 1, further comprising a solution presence indicator operably coupled to the sterilization applicator.

8. A puncture preparation device comprising:
(a) a body;
(b) a skin marking mechanism disposed at a first end of the body;
(c) a removable cover removably disposable over the skin marking device, the removable cover comprising a skin marking feature disposed thereon;
(d) a sterilization application structure disposed at a second end of the body; and
(e) a fracturable sterilization solution reservoir disposed in the body.

9. The puncture preparation device of claim 8, wherein the body comprises at least one flexible portion disposed between a first portion of the body and a second portion of the body, wherein the first portion is movable relation to the second portion of the body at the at least one flexible portion.

10. The puncture preparation device of claim 9, wherein movement of the first portion in relation to the second portion causes the fracturable sterilization solution reservoir to break such that sterilization solution disposed therein can flow into the sterilization application structure.

11. The puncture preparation device of claim 9, further comprising an actuation mechanism operably coupled with the body, wherein actuation of the actuation mechanism causes the fracturable sterilization solution reservoir to fracture.

12. The puncture preparation device of claim 8, wherein the skin marking feature comprises at least one raised feature on a distal end of the removable cover.

13. The puncture preparation device of claim 12, wherein the at least one raised feature is configured to create an indentation in a patient's skin when sufficient force is applied.

14. The puncture preparation device of claim 8, further comprising a solution presence indicator operably coupled to the sterilization application structure.

15. The puncture preparation device of claim 8, further comprising a filter disposed between the fracturable sterilization solution reservoir and the sterilization application structure.

16. A method of preparing a target site on a patient's skin for a needle penetration, the method comprising:
positioning a needle penetration preparation device with a single hand of a user such that a skin marking mechanism is disposed in proximity with the target site, the device comprising:
(a) a device body;
(b) the skin marking mechanism disposed at a first end of the device body;
(c) a removable cap removably coupled to the skin marking device, the removable cap comprising a skin marking feature disposed on the removable cap;
(d) a sterilization applicator disposed at a second end of the device body; and
(e) a sterilization solution reservoir disposed in the body, wherein the reservoir is adjacent to the sterilization applicator;
marking the target site with the skin marking mechanism or the skin marking feature;
repositioning the device within the single hand of the user such that the sterilization applicator is disposed in proximity with the target site;
causing the sterilization solution reservoir to be compromised such that the sterilization solution flows out of the reservoir and into the sterilization applicator; and
applying the sterilization solution to the target site with the sterilization applicator.

17. The method of claim 16, wherein the causing the sterilization solution reservoir to be compromised further comprises deforming the device body such that the reservoir is compromised.

18. The method of claim 16, wherein the causing the sterilization solution reservoir to be compromised further comprises actuating an actuation mechanism such that the reservoir is compromised.

19. The method of claim 16, further comprising removing the removable cap from the skin marking mechanism prior to marking the target site with the skin marking mechanism.

20. The method of claim 16, wherein the applying the sterilization solution further comprises applying the sterilization solution for a minimum amount of time based on an indicator operably coupled to the sterilization applicator.
